(19) United States
(12) Patent Application Publication
(10) Pub. No.: US 2025/0295665 A1
(43) Pub. Date: Sep. 25, 2025

(54) SELECTIVE K-RAS INHIBITORS FOR THE ENHANCEMENT OF ADOPTIVE IMMUNOTHERAPIES

(71) Applicant: H. LEE MOFFITT CANCER CENTER AND RESEARCH INSTITUTE INC., Tampa, FL (US)

(72) Inventors: Amer BEG, Tampa, FL (US); Daniel ABATE-DAGA, Tampa, FL (US)

(21) Appl. No.: 18/863,039

(22) PCT Filed: May 10, 2023
(86) PCT No.: PCT/US2023/066824

§ 371 Date: Nov. 5, 2024

Related U.S. Application Data
(60) Provisional application No. 63/364,447, filed on May 10, 2022.

(51) Int. Cl. A61K 31/519; A61K 35/17; A61K 40/11; A61K 40/31; A61P 35/00; C07K 16/28

(52) U.S. Cl. CPC A61K 31/519; A61K 35/17; A61K 40/11; A61K 40/31; A61P 35/00; C07K 16/2818; C07K 16/2827

(57) ABSTRACT

Sotorasib augments the KRAS-G12C mutant cancer cells response to TNFα by Increasing TNFR1 surface expression, which in turn upregulates TNFα and IFNy down-stream target genes and ultimately enhances cancer cell death. In TACE-dependent mechanism, Sotorasib inhibited TNFR1 shedding off the KRAS-G12C mutant cancer cells. Additionally, Sotorasib significantly promotes the expansion of tumor infiltrating lymphocytes (TILs). In, addition, it generates a “hot” tumor microenvironment with strikingly augmented T cell effector phenotype. These results suggest that sotorasib regulation of TNFa and IFNy plays a crucial role in generating a more immune active tumor microenvironment. Consistently, sotorasib combined with CAR-T adoptive cell transfer and anti-PD1 treatment enhanced their anti-tumor effect. Therefore, disclosed herein is a method for treating KRAS-G12C mutant cancer in a subject, the method involving adoptively transferring an effective amount of autologous or allogeneic immune effector cells (T cells) to the subject in combination with a KRAS-G12C inhibitor

### CROSS-REFERENCE TO RELATED APPLICATIONS

This application claims benefit of U.S. Provisional Application No. 63/364,447, filed May 10, 2023, which is hereby incorporated herein by reference in its entirety.

### STATEMENT OF GOVERNMENT INTEREST

This invention was made with Government Support under Grant No. W81XWH-15-1-0376 awarded by the Department of Defense. The Government has certain rights in the invention.

### BACKGROUND OF THE INVENTION

KRAS-G12C mutation represents a highly prevalent mutation in lung and colon carcinoma with unfavorable clinical outcome. Recently, new compounds were developed that selectively bind to K-RAS with G12C mutation. While recent clinical results demonstrate activity of these compounds, their underlying mechanisms of action and impact on the antitumor immune response are not well understood.

### SUMMARY OF THE INVENTION

As disclosed herein, sotorasib augments the KRAS-G12C mutant cancer cells response to TNFα by increasing TNFR1 surface expression, which in turn upregulates TNFα and IFNγ down-stream target genes (including T cells chemokines) and ultimately enhances cancer cell death. In TACE-dependent mechanism, Sotorasib inhibited TNFR1 shedding off the KRAS-G12C mutant cancer cells. Interestingly, Sotorasib significantly promoted the expansion of tumor infiltrating lymphocytes (TILs) in both CT26-G12C heterotrophic, and human T cell adoptive therapy (ACT) mouse models. In addition, it generated a “hot” tumor microenvironment with strikingly augmented T cell effector phenotype. Intriguingly, Single cell RNA sequencing of CT26-G12C tumors showed an enrichment of tumor clusters with TNFα and IFNγ as top upregulated pathways in Sotorasib-treated tumors. These results suggest that Sotorasib regulation of TNFα and IFNγ plays a crucial role in generating a more immune active TME. Consistently, Sotorasib synergized with anti-PD-1 in controlling CT26-G12C tumor growth and prolonging mouse survival. Similarly, Sotorasib combined with CAR-T adoptive cell transfer and anti-PD1 treatment enhanced their anti-tumor effect.

These findings provide insight into the molecular basis of G12C-Kras inhibitor-mediated immune modulation in the TME and set the foundation for combinatorial regimens with checkpoint inhibitor and ACT immunotherapy to achieve optimum anti-tumor activity.

Therefore, disclosed herein is a method for treating KRAS-G12C mutant cancer in a subject, the method involving adoptively transferring an effective amount of autologous or allogeneic immune effector cells (T cells) to the subject.

In some embodiments, the immune effector cell is selected from the group consisting of an alpha-beta T cells, a gamma-delta T cell, a Natural Killer (NK) cells, a Natural Killer T (NKT) cell, a B cell, an innate lymphoid cell (ILC), a cytokine induced killer (CIK) cell, a cytotoxic T lymphocyte (CTL), a lymphokine activated killer (LAK) cell, tumor infiltrating lymphocyte (TIL), and a regulatory T cell.

Also disclosed is a method for enhancing adoptive cell transfer (ACT) treatment of an immune effector cell in a subject, the method comprising co-administering to the subject a KRAS inhibitor.

In some embodiments of the disclosed methods, the KRAS-G12C is administered simultaneously with the immune effector cell. In other embodiments, the KRAS-G12C is administered daily for at least 1, 2, 3, 4, 5, 6, or 7 days or 1, 2, 3, or 4 weeks prior to the immune effector cell.

The details of one or more embodiments of the invention are set forth in the accompanying drawings and the description below. Other features, objects, and advantages of the invention will be apparent from the description and drawings, and from the claims.

### BRIEF DESCRIPTION OF FIGURES

FIGS. 1A to 1G show KRAS G12C inhibitor impact on TNFα and IFNγ pathways. FIG. 1A shows HCC44 (G12C-Kras mutant) and A549 (G12V-Kras Mutant) lung cancer cell lines treated with TRA (10 nM) or AMG-510 (100 nM) for 1 day then stained with anti-h TNFR1 antibody and analyzed by flow cytometry. In FIGS. 1B, 1C, 1D, and 1G HCC44, HCC44-TNFR1KO and H23, and CRISPR edited G12C-CT26 cell lines were treated with the indicated condition, for CXCL10 mRNA expression levels by qPCR. FIG. 1E shows HCC44 cells treated with the indicated condition for 3 days. Cells were then harvested, stained with trypan blue and counted for viability. all conditions were normalized to the control. G12C-CT26 colon cancer cells were seeded in ULA 96 well plate to form 3D spheroids, then treated with the indicated conditions and analyzed for viability using 3D CTG viability assay.

FIGS. 2A to 2G show sotorasib upregulates TNFR1 surface expression in a TACE-dependent mechanism. FIGS. 2A, 2B, and 2C show cells treated with TRA (10 nm), AMG-510 (100 nM) or TAPI (200 uM) for 24 hours. Then soluble TNFR1 measured from CM by ELISA. Cells were lysed and analyzed for TACE activity. FIG. 2D shows secreted TNF-α from macrophages induced from CD14-isolated Monocytes was measured by ELISA. FIGS. 2E, 2F, and 2G show CD3-Isolated T cells from buffy coat PBMC treated with TRA or AMG, then measure TNFα secretion by ELISA or Surface TNFR1, TNFR2 by Flow cytometry.

FIGS. 3A to 3L show sotorasib enhances T-cells infiltration and function in TME and synergize with anti-PD1. CT26-G12C colon cancer cells were SC. Implanted in BALB/C mice, and left to develop SC. Tumors for 20 days, then treated with oral gavage of 3 mg/kg/mouse TRA or 30 mg/kg/mouse AMG-510 for 6 days alone or for 20 days with anti-PD1 Ab (IP 200 μg/mouse twice a week). Tumor size was measured till mice being sacrificed. tumors were collected and stained for T cell, and Myeloid cell markers mouse antibodies (anti-CD45, anti-CD8, anti-CD4, anti-CD3, anti-CD69, anti-PD1, anti-CD25, anti-CD62L, anti-CD44, anti-F4/80, anti-CD11C, anti-CD11b, anti-Ly6G, ant-Ly6C and Live/Dead near IR viability dye). CM (central memory): CD62L+CD44+, EM (Effector memory) CD62L-CD44+. Mice survival was assessed using Kaplan-Meier survival test.

FIGS. 4A to 4F show sotorasib enriches the immuno-active and diminishes growth-active tumor clusters. UMAP projections were generated from scRNA-seq data and used for visualization. Cell type clusters detected in DAPI-sorted cells from CT26-G12C SC. Tumors (FIG. 4A), or from CT26-G12C in vitro cell line (FIG. 4D) treated with the indicated conditions are shown, with cell % percentage histogram and gene-level expression heat map calculated for each cluster.

FIGS. 5A and 5B show sotorasib promotes the anti-tumor effect of CAR-T ACT and anti-PD1 ICI. FIG. 5A shows PSCA-expressing HCC44 and H23 cells were cocultured with PSCA-Specific CAR-T, or parental cells were exposed to CM collected from CAR-T with PSCA expressing cells and treated with the indicated conditions then measure viability by Luciferase Assay. FIG. 5B shows HCC44-PSCA were SC. Inoculated in NSG mice and received IV CART transfer with AMG-510 Orla gavage (30 mg/Kg/Mouse for 6 days then sacrificed and tumors were collected and stained with T cells markers, or for 20 days with IP anti-human PD1 Ab (twice a week) for Tumor growth curve.

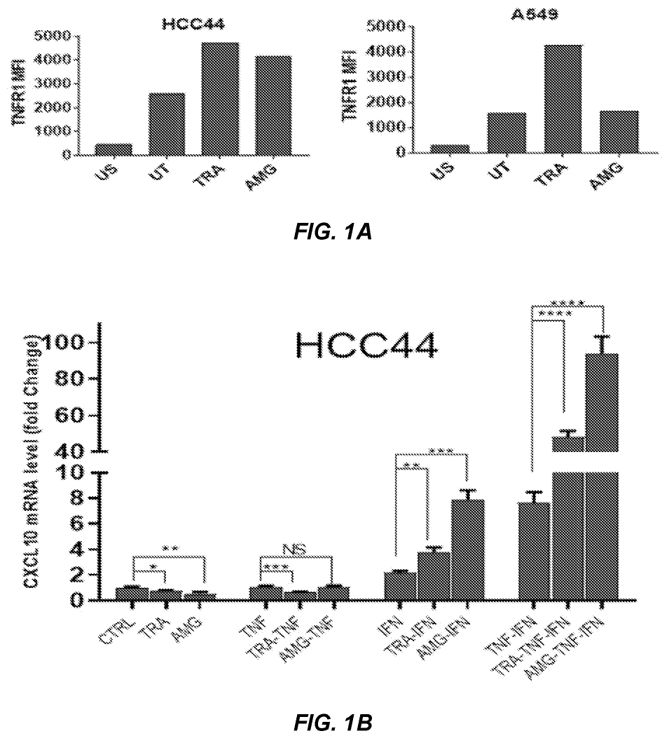

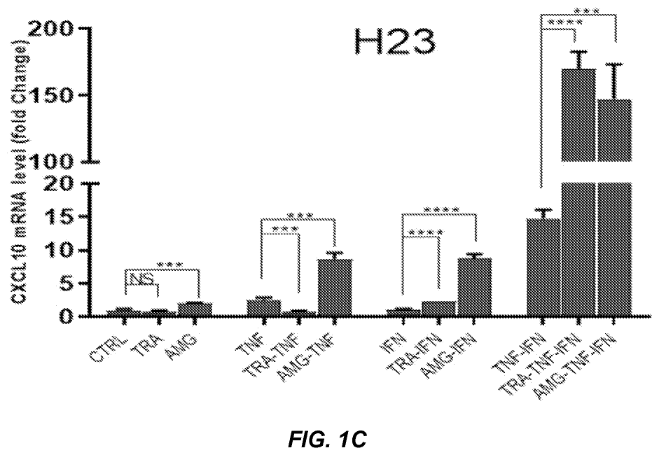

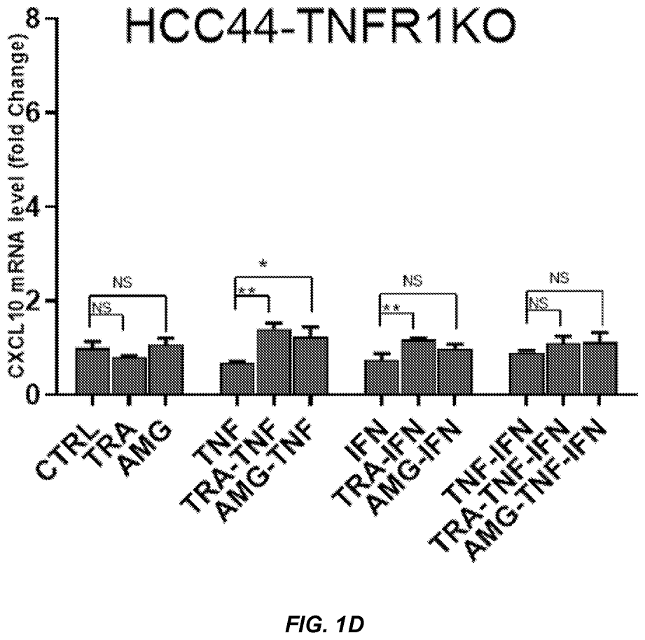

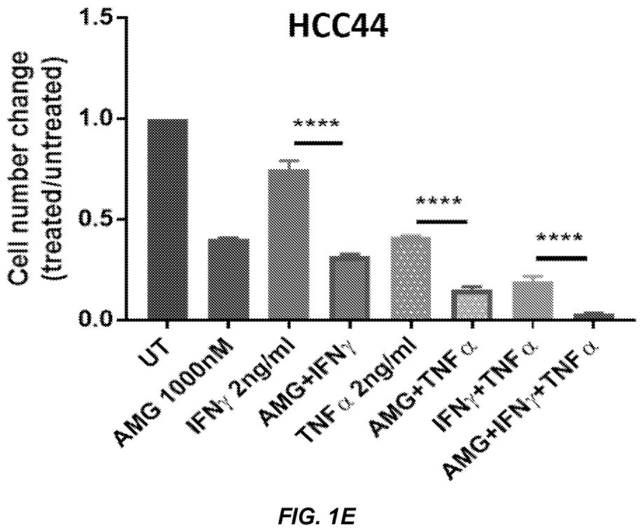

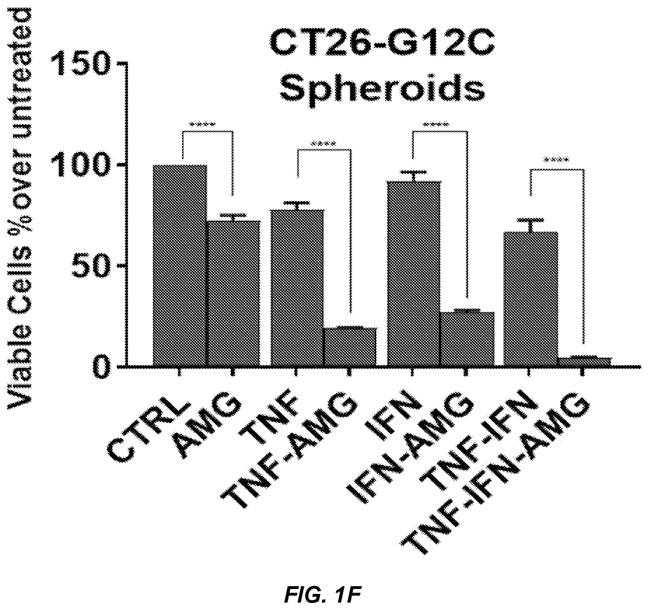

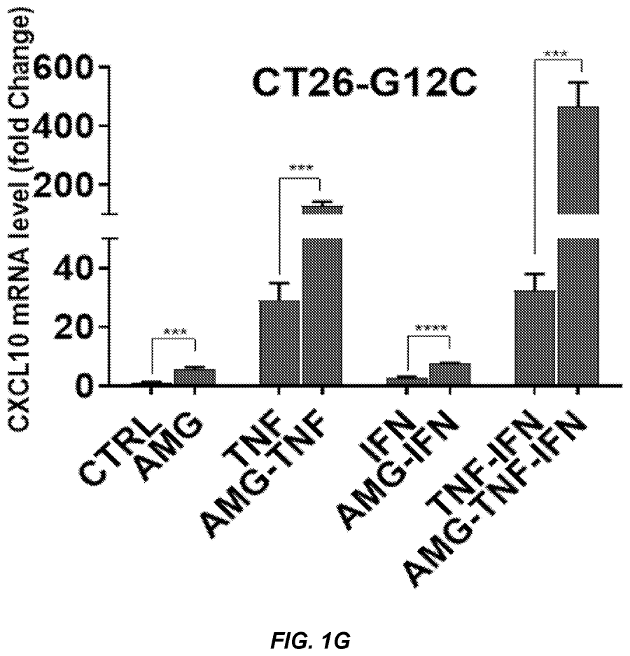

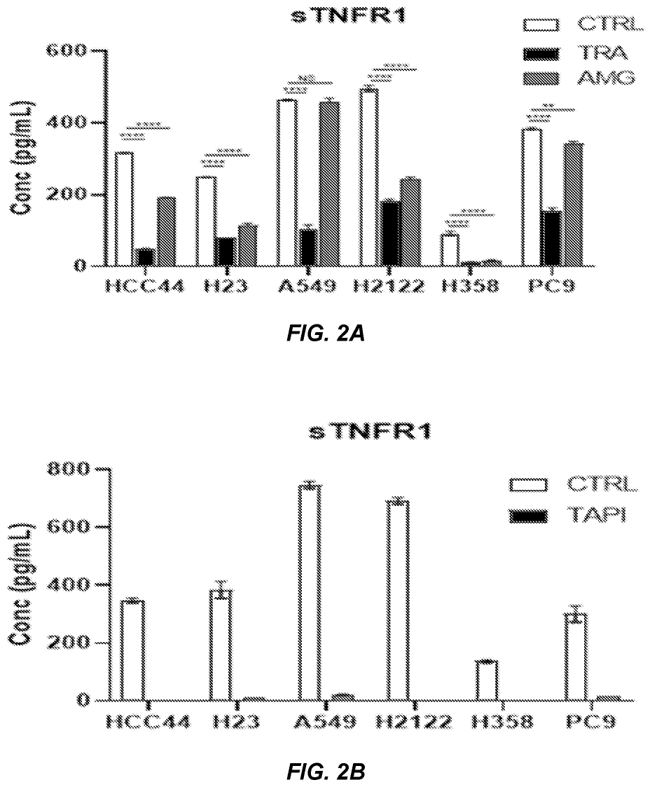

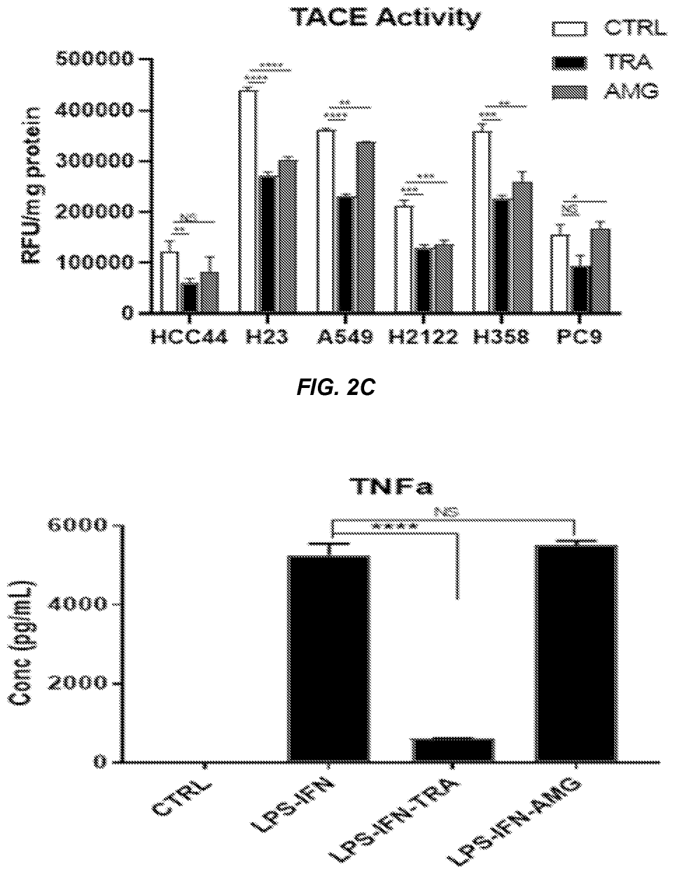

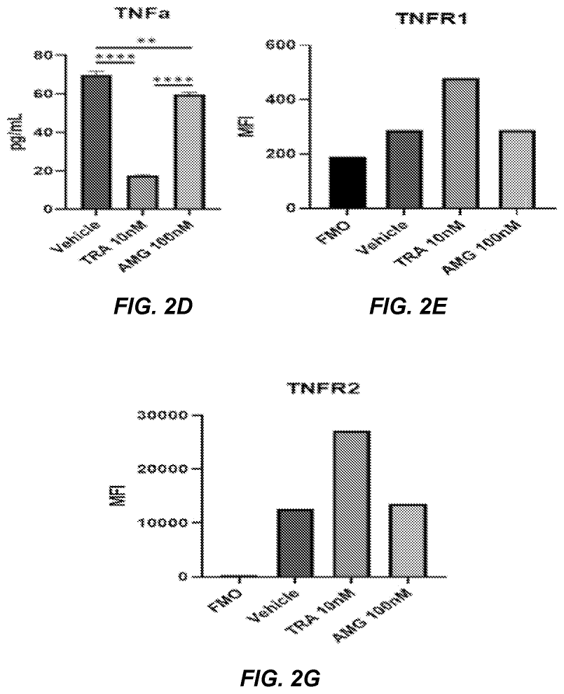

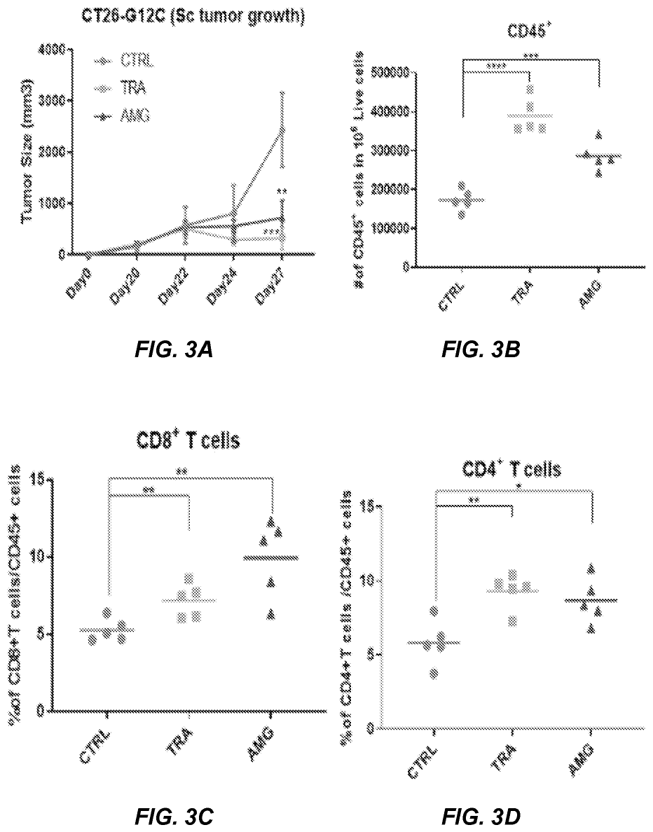

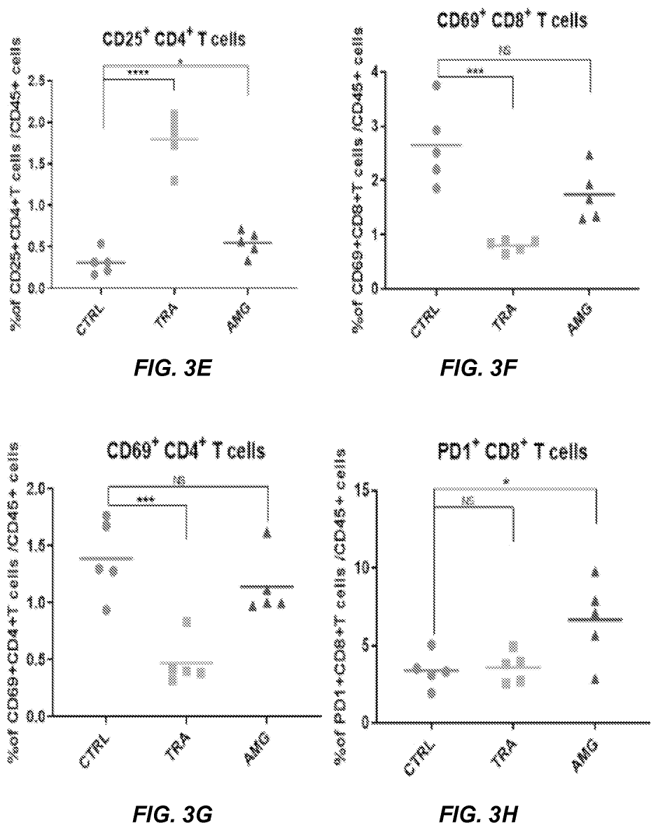

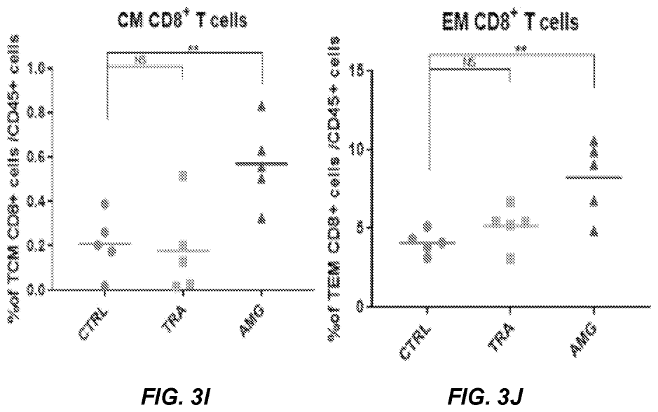

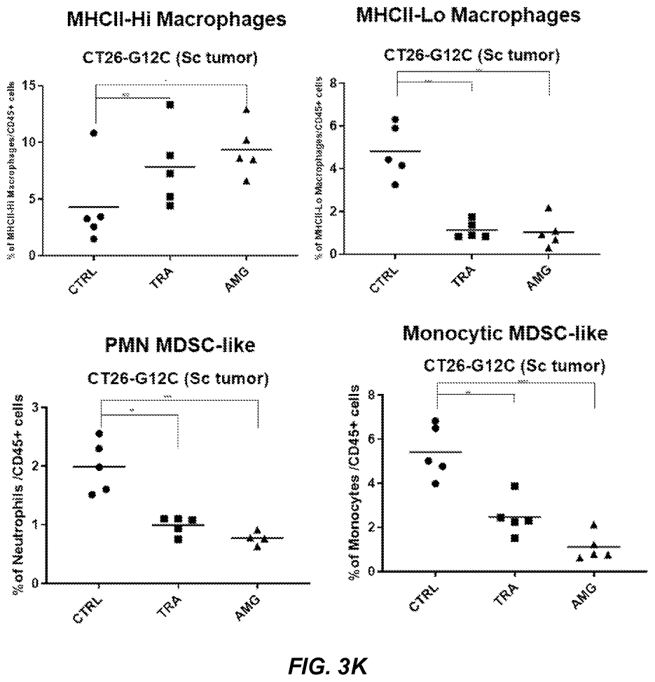

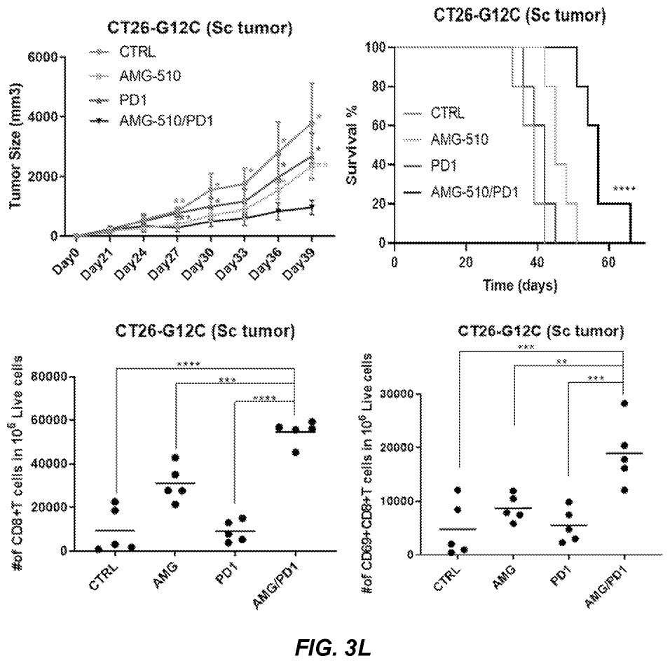

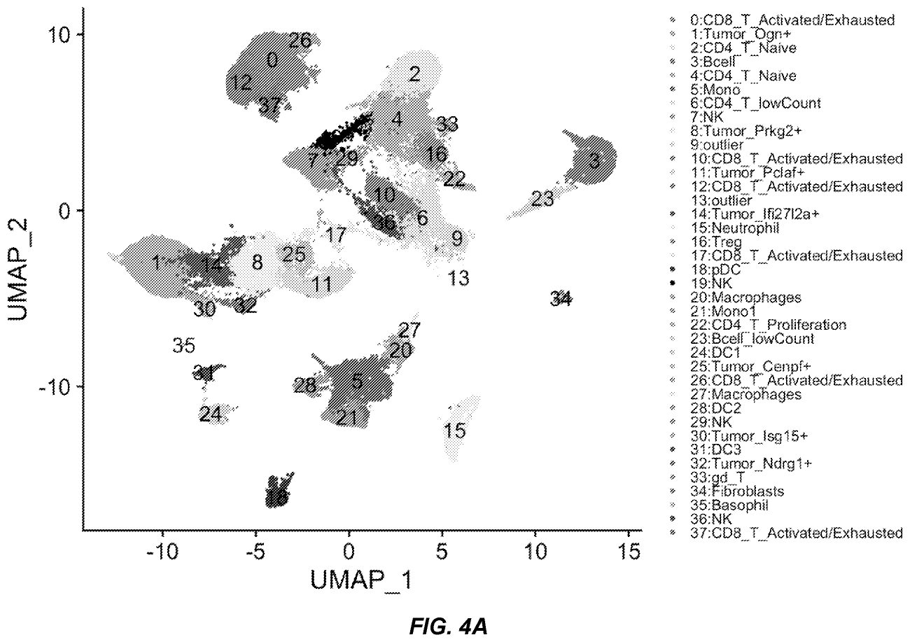

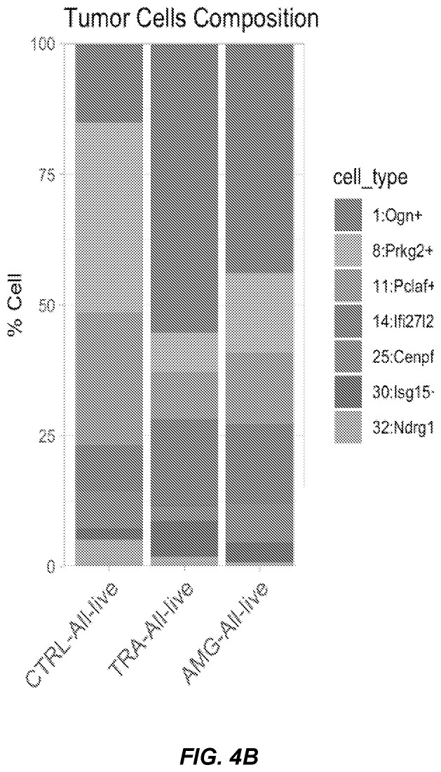

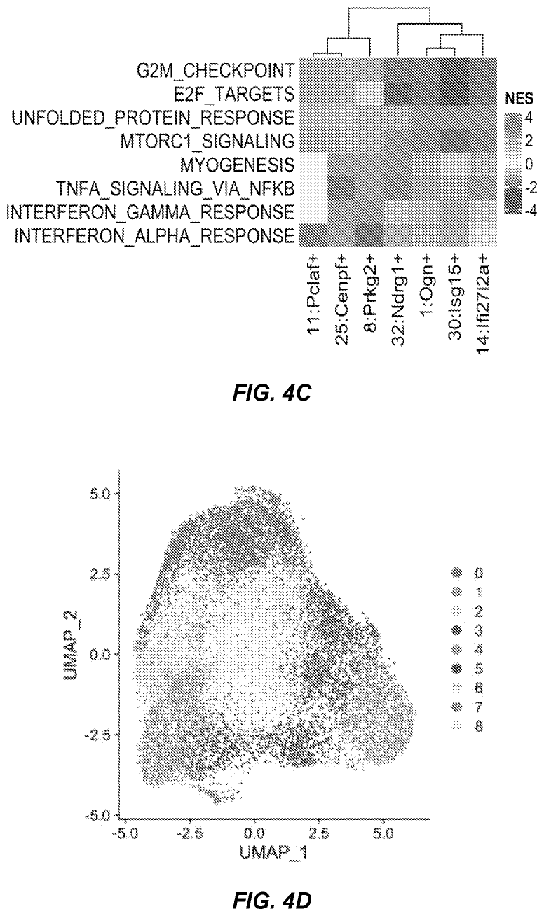

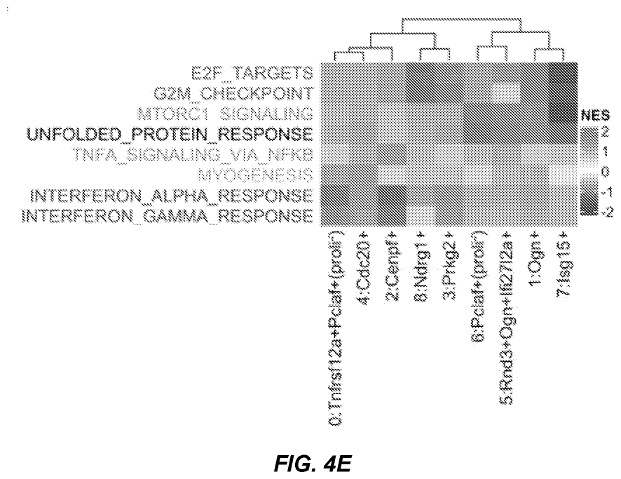

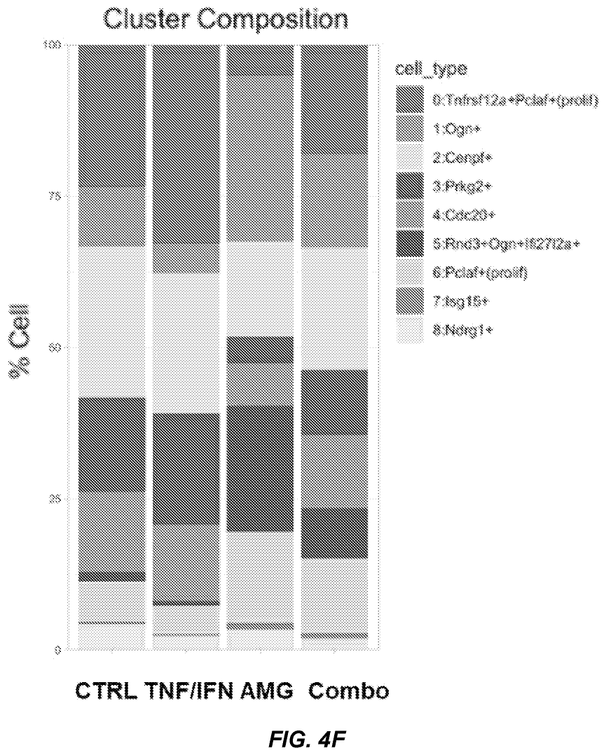

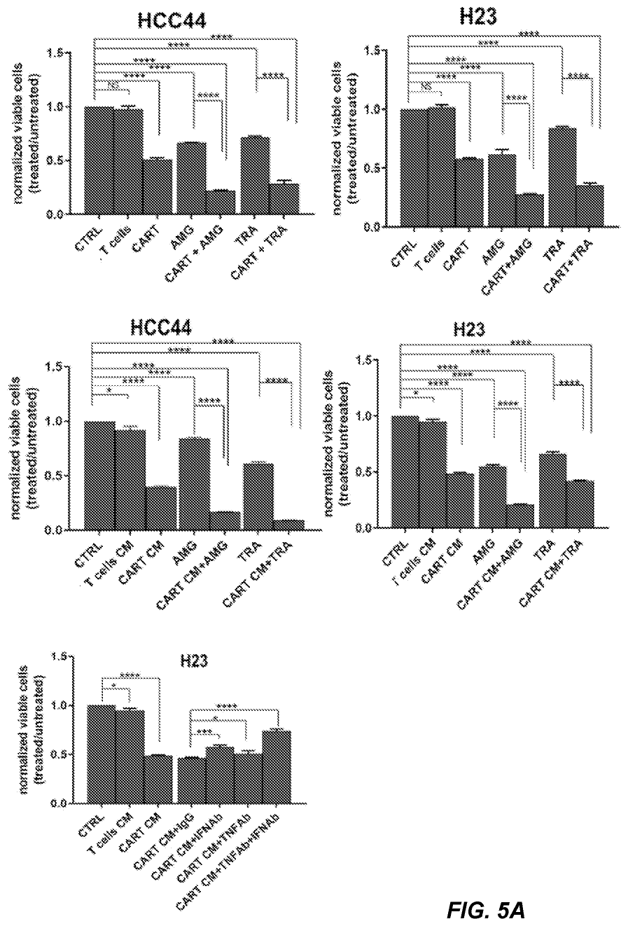

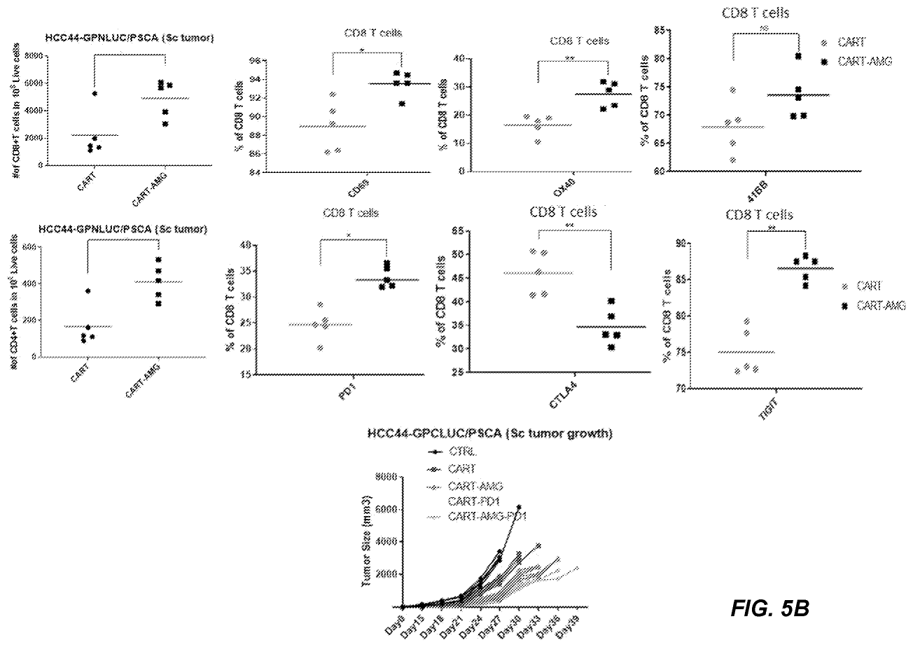

### DETAILED DESCRIPTION

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting, since the scope of the present disclosure will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of chemistry, biology, and the like, which are within the skill of the art.

The following examples are put forth so as to provide those of ordinary skill in the art with a complete disclosure and description of how to perform the methods and use the probes disclosed and claimed herein. Efforts have been made to ensure accuracy with respect to numbers (e.g., amounts, temperature, etc.), but some errors and deviations should be accounted for. Unless indicated otherwise, parts are parts by weight, temperature is in ° C., and pressure is at or near atmospheric. Standard temperature and pressure are defined as 20° C. and 1 atmosphere.

Before the embodiments of the present disclosure are described in detail, it is to be understood that, unless otherwise indicated, the present disclosure is not limited to particular materials, reagents, reaction materials, manufacturing processes, or the like, as such can vary. It is also to be understood that the terminology used herein is for purposes of describing particular embodiments only, and is not intended to be limiting. It is also possible in the present disclosure that steps can be executed in different sequence where this is logically possible.

It must be noted that, as used in the specification and the appended claims, the singular forms “a,” “an,” and “the” include plural referents unless the context clearly dictates otherwise.

The term “subject” refers to any individual who is the target of administration or treatment. The subject can be a vertebrate, for example, a mammal. Thus, the subject can be a human or veterinary patient. The term “patient” refers to a subject under the treatment of a clinician, e.g., physician.

The term “therapeutically effective” refers to the amount of the composition used is of sufficient quantity to ameliorate one or more causes or symptoms of a disease or disorder. Such amelioration only requires a reduction or alteration, not necessarily elimination.

The term “pharmaceutically acceptable” refers to those compounds, materials, compositions, and/or dosage forms which are, within the scope of sound medical judgment, suitable for use in contact with the tissues of human beings and animals without excessive toxicity, irritation, allergic response, or other problems or complications commensurate with a reasonable benefit/risk ratio.

The term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder.

### Kras Inhibitors

In some embodiments, the KRAS inhibitor is selected from the group consisting of AMG 510 (sotorasib, LUMAKRAS™), MRTX849 (adagrasib), ARS-3248, GDC-6036, BI 1701963, tipifarnib and BBP-454.

Sotorasib is a small molecule that irreversibly inhibits the KRAS^{G12C} mutant protein. Sotorasib is also referred to as AMG 510 or 6-fluoro-7-(2-fluoro-6-hydroxyphenyl)-(1/W)-1-[4-methyl-2-(propan-2-yl) pyridin-3-yl]-4-[(2S)-2-methyl-4-(prop-2-enoyl) piperazin-1-yl]pyrido[2,3-cf]pyrimidin-2 (1/-/)-one and has the following structure:

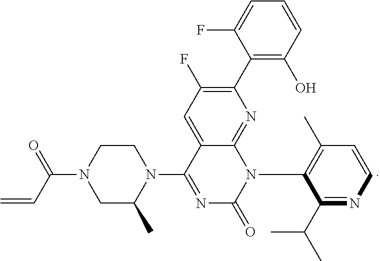

Sotorasib binds to the P2 pocket of KRAS adjacent to the mutant cysteine at position 12 and the nucleotide-binding pocket. The inhibitor contains a thiol reactive portion which covalently modifies the cysteine residue and locks KRAS^{G12C} in an inactive, guanosine diphosphate (GDP) bound conformation. This blocks the interaction of KRAS with effectors such as rapidly accelerated fibrosarcoma (RAF), thereby preventing downstream signaling, including the phosphorylation of extracellular signal regulated kinase (ERK) (Cully and Downward, 2008; Ostrem et al., 2013; Simanshu et al., 2017). Inactivation of KRAS by RNA interference (RNAi) or small molecule inhibition has previously demonstrated an inhibition of cell growth and induction of apoptosis in tumor cell lines and xenografts harboring KRAS mutations (including the KRAS G12C mutation) (Janes et al., 2018; McDonald et al., 2017; Xie et al., 2017; Ostrem and Shokat, 2016; Patricelli et al., 2016). Studies with sotorasib have confirmed these in vitro findings and have likewise demonstrated inhibition of growth and regression of cells and tumors harboring KRAS G12C mutations (Canon et al., 2019). See also, LUMAKRAS® US Prescribing Information, Amgen Inc., Thousand Oaks, California, 91320 (revision May 2021), which is herein incorporated by reference in its entirety.

### Chimeric Antigen Receptors (CAR)

In particular embodiments, the immune effector cells with ablated KIR2DL2 are engineered to express a chimeric antigen receptor (CAR) polypeptide. CARs generally incorporate an antigen recognition domain from the single-chain variable fragments (scFv) of a monoclonal antibody (mAb) with transmembrane signaling motifs involved in lymphocyte activation (Sadelain M, et al. Nat Rev Cancer 2003 3:35-45). The disclosed CAR is generally made up of three domains: an ectodomain, a transmembrane domain, and an endodomain. The ectodomain comprises the recognition domain. It also optionally contains a signal peptide (SP) so that the CAR can be glycosylated and anchored in the cell membrane of the immune effector cell. The transmembrane domain (TD), as its name suggests, connects the ectodomain to the endodomain and resides within the cell membrane when expressed by a cell. The endodomain is the business end of the CAR that transmits an activation signal to the immune effector cell after antigen recognition. For example, the endodomain can contain an intracellular signaling domain (ISD) and optionally a co-stimulatory signaling region (CSR).

A “signaling domain (SD)” generally contains immunoreceptor tyrosine-based activation motifs (ITAMs) that activate a signaling cascade when the ITAM is phosphorylated. The term “co-stimulatory signaling region (CSR)” refers to intracellular signaling domains from costimulatory protein receptors, such as CD28, 41BB, and ICOS, that are able to enhance T-cell activation by T-cell receptors.

In some embodiments, the endodomain contains an SD or a CSR, but not both. In these embodiments, an immune effector cell containing the disclosed CAR is only activated if another CAR (or a T-cell receptor) containing the missing domain also binds its respective antigen.

Additional CAR constructs are described, for example, in Fresnak A D, et al. Engineered T cells: the promise and challenges of cancer immunotherapy. Nat Rev Cancer. 2016 Aug. 23; 16 (9): 566-81, which is incorporated by reference in its entirety for the teaching of these CAR models.

For example, the CAR can be a TRUCK, Universal CAR, Self-driving CAR, Armored CAR, Self-destruct CAR, Conditional CAR, Marked CAR, TenCAR, Dual CAR, or SCAR.

TRUCKs (T cells redirected for universal cytokine killing) co-express a chimeric antigen receptor (CAR) and an antitumor cytokine. Cytokine expression may be constitutive or induced by T cell activation. Targeted by CAR specificity, localized production of pro-inflammatory cytokines recruits endogenous immune cells to tumor sites and may potentiate an antitumor response.

Universal, allogeneic CAR T cells are engineered to no longer express endogenous T cell receptor (TCR) and/or major histocompatibility complex (MHC) molecules, thereby preventing graft-versus-host disease (GVHD) or rejection, respectively.

Self-driving CARs co-express a CAR and a chemokine receptor, which binds to a tumor ligand, thereby enhancing tumor homing.

CAR T cells engineered to be resistant to immunosuppression (Armored CARs) may be genetically modified to no longer express various immune checkpoint molecules (for example, cytotoxic T lymphocyte-associated antigen 4 (CTLA4) or programmed cell death protein 1 (PD1)), with an immune checkpoint switch receptor, or may be administered with a monoclonal antibody that blocks immune checkpoint signaling.

A self-destruct CAR may be designed using RNA delivered by electroporation to encode the CAR. Alternatively, inducible apoptosis of the T cell may be achieved based on ganciclovir binding to thymidine kinase in gene-modified lymphocytes or the more recently described system of activation of human caspase 9 by a small-molecule dimerizer.

A conditional CAR T cell is by default unresponsive, or switched ‘off’, until the addition of a small molecule to complete the circuit, enabling full transduction of both signal 1 and signal 2, thereby activating the CAR T cell. Alternatively, T cells may be engineered to express an adaptor-specific receptor with affinity for subsequently administered secondary antibodies directed at target antigen.

Marked CAR T cells express a CAR plus a tumor epitope to which an existing monoclonal antibody agent binds. In the setting of intolerable adverse effects, administration of the monoclonal antibody clears the CAR T cells and alleviates symptoms with no additional off-tumor effects.

A tandem CAR (TanCAR) T cell expresses a single CAR consisting of two linked single-chain variable fragments (scFvs) that have different affinities fused to intracellular co-stimulatory domain(s) and a CD3ζ domain. TanCAR T cell activation is achieved only when target cells co-express both targets.

A dual CAR T cell expresses two separate CARs with different ligand binding targets; one CAR includes only the CD3ζ domain and the other CAR includes only the co-stimulatory domain(s). Dual CAR T cell activation requires co-expression of both targets on the tumor.

A safety CAR (sCAR) consists of an extracellular scFv fused to an intracellular inhibitory domain. SCAR T cells co-expressing a standard CAR become activated only when encountering target cells that possess the standard CAR target but lack the sCAR target.

The antigen recognition domain of the disclosed CAR is usually an scFv. There are however many alternatives. An antigen recognition domain from native T-cell receptor (TCR) alpha and beta single chains have been described, as have simple ectodomains (e.g. CD4 ectodomain to recognize HIV infected cells) and more exotic recognition components such as a linked cytokine (which leads to recognition of cells bearing the cytokine receptor). In fact almost anything that binds a given target with high affinity can be used as an antigen recognition region.

The endodomain is the business end of the CAR that after antigen recognition transmits a signal to the immune effector cell, activating at least one of the normal effector functions of the immune effector cell. Effector function of a T cell, for example, may be cytolytic activity or helper activity including the secretion of cytokines. Therefore, the endodomain may comprise the “intracellular signaling domain” of a T cell receptor (TCR) and optional co-receptors. While usually the entire intracellular signaling domain can be employed, in many cases it is not necessary to use the entire chain. To the extent that a truncated portion of the intracellular signaling domain is used, such truncated portion may be used in place of the intact chain as long as it transduces the effector function signal.

Cytoplasmic signaling sequences that regulate primary activation of the TCR complex that act in a stimulatory manner may contain signaling motifs which are known as immunoreceptor tyrosine-based activation motifs (ITAMs). Examples of ITAM containing cytoplasmic signaling sequences include those derived from CD8, CD3ζ, CD3δ, CD3γ, CD3ε, CD32 (Fc gamma RIIa), DAP10, DAP12, CD79a, CD79b, FcγRIγ, FcγRIIIγ, FcεRIβ (FCERIB), and FcεRIγ (FCERIG).

In particular embodiments, the intracellular signaling domain is derived from CD3 zeta (CD3ζ) (TCR zeta, GenBank accno. BAG36664.1). T-cell surface glycoprotein CD3 zeta (CD3ζ) chain, also known as T-cell receptor T3 zeta chain or CD247 (Cluster of Differentiation 247), is a protein that in humans is encoded by the CD247 gene.

First-generation CARs typically had the intracellular domain from the CD3ζ chain, which is the primary transmitter of signals from endogenous TCRs. Second-generation CARs add intracellular signaling domains from various costimulatory protein receptors (e.g., CD28, 41BB, ICOS) to the endodomain of the CAR to provide additional signals to the T cell. Preclinical studies have indicated that the second generation of CAR designs improves the antitumor activity of T cells. More recent, third-generation CARs combine multiple signaling domains to further augment potency. T cells grafted with these CARs have demonstrated improved expansion, activation, persistence, and tumor-eradicating efficiency independent of costimulatory receptor/ligand interaction (Imai C, et al. Leukemia 2004 18:676-84; Maher J, et al. Nat Biotechnol 2002 20:70-5).

For example, the endodomain of the CAR can be designed to comprise the CD3ζ signaling domain by itself or combined with any other desired cytoplasmic domain(s) useful in the context of the CAR of the invention. For example, the cytoplasmic domain of the CAR can comprise a CD3ζ chain portion and a costimulatory signaling region. The costimulatory signaling region refers to a portion of the CAR comprising the intracellular domain of a costimulatory molecule. A costimulatory molecule is a cell surface molecule other than an antigen receptor or their ligands that is required for an efficient response of lymphocytes to an antigen. Examples of such molecules include CD27, CD28, 4-1BB (CD137), OX40, CD30, CD40, ICOS, lymphocyte function-associated antigen-1 (LFA-1), CD2, CD7, LIGHT, NKG2C, B7-H3, and a ligand that specifically binds with CD83, CD8, CD4, b2c, CD80, CD86, DAP10, DAP12, MyD88, BTNL3, and NKG2D. Thus, while the CAR is exemplified primarily with CD28 as the co-stimulatory signaling element, other costimulatory elements can be used alone or in combination with other co-stimulatory signaling elements.

In some embodiments, the CAR comprises a hinge sequence. A hinge sequence is a short sequence of amino acids that facilitates antibody flexibility (see, e.g., Woof et al., Nat. Rev. Immunol., 4(2): 89-99 (2004)). The hinge sequence may be positioned between the antigen recognition moiety (e.g., anti-CD123 scFv) and the transmembrane domain. The hinge sequence can be any suitable sequence derived or obtained from any suitable molecule. In some embodiments, for example, the hinge sequence is derived from a CD8a molecule or a CD28 molecule.

The transmembrane domain may be derived either from a natural or from a synthetic source. Where the source is natural, the domain may be derived from any membrane-bound or transmembrane protein. For example, the transmembrane region may be derived from (i.e. comprise at least the transmembrane region(s) of) the alpha, beta or zeta chain of the T-cell receptor, CD28, CD3 epsilon, CD45, CD4, CD5, CD8 (e.g., CD8 alpha, CD8 beta), CD9, CD16, CD22, CD33, CD37, CD64, CD80, CD86, CD134, CD137, or CD154, KIRDS2, OX40, CD2, CD27, LFA-1 (CD11a, CD18), ICOS (CD278), 4-1BB (CD137), GITR, CD40, BAFFR, HVEM (LIGHTR), SLAMF7, NKp80 (KLRF1), CD160, CD19, IL2R beta, IL2R gamma, IL7R α, ITGA1, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, TNFR2, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), SLAMF6 (NTB-A, Ly108), SLAM (SLAMF1, CD150, IPO-3), BLAME (SLAMF8), SELPLG (CD162), LTBR, and PAG/Cbp. Alternatively the transmembrane domain may be synthetic, in which case it will comprise predominantly hydrophobic residues such as leucine and valine. In some cases, a triplet of phenylalanine, tryptophan and valine will be found at each end of a synthetic transmembrane domain. A short oligo- or polypeptide linker, such as between 2 and 10 amino acids in length, may form the linkage between the transmembrane domain and the endoplasmic domain of the CAR.

In some embodiments, the CAR has more than one transmembrane domain, which can be a repeat of the same transmembrane domain, or can be different transmembrane domains.

In some embodiments, the CAR is a multi-chain CAR, as described in WO2015/039523, which is incorporated by reference for this teaching. A multi-chain CAR can comprise separate extracellular ligand binding and signaling domains in different transmembrane polypeptides. The signaling domains can be designed to assemble in juxtamembrane position, which forms flexible architecture closer to natural receptors, that confers optimal signal transduction. For example, the multi-chain CAR can comprise a part of an FCERI alpha chain and a part of an FCERI beta chain such that the FCERI chains spontaneously dimerize together to form a CAR.

In some embodiments, the recognition domain is a single chain variable fragment (scFv) antibody. The affinity/specificity of an scFv is driven in large part by specific sequences within complementarity determining regions (CDRs) in the heavy (V_{H}) and light (V_{L}) chain. Each V_{H} and V_{L} sequence will have three CDRs (CDR1, CDR2, CDR3).

In some embodiments, the recognition domain is derived from natural antibodies, such as monoclonal antibodies. In some cases, the antibody is human. In some cases, the antibody has undergone an alteration to render it less immunogenic when administered to humans. For example, the alteration comprises one or more techniques selected from the group consisting of chimerization, humanization, CDR-grafting, deimmunization, and mutation of framework amino acids to correspond to the closest human germline sequence.

Also disclosed are bi-specific CARs that target two different antigens. Also disclosed are CARs designed to work only in conjunction with another CAR that binds a different antigen, such as a tumor antigen. For example, in these embodiments, the endodomain of the disclosed CAR can contain only a signaling domain (SD) or a co-stimulatory signaling region (CSR), but not both. The second CAR (or endogenous T-cell) provides the missing signal if it is activated. For example, if the disclosed CAR contains an SD but not a CSR, then the immune effector cell containing this CAR is only activated if another CAR (or T-cell) containing a CSR binds its respective antigen. Likewise, if the disclosed CAR contains a CSR but not a SD, then the immune effector cell containing this CAR is only activated if another CAR (or T-cell) containing an SD binds its respective antigen.

Tumor antigens are proteins that are produced by tumor cells that elicit an immune response, particularly T-cell mediated immune responses. The additional antigen binding domain can be an antibody or a natural ligand of the tumor antigen. The selection of the additional antigen binding domain will depend on the particular type of cancer to be treated. Tumor antigens are well known in the art and include, for example, a glioma-associated antigen, carcinoembryonic antigen (CEA), EGFRvIII, IL-IIRa, IL-13Ra, EGFR, FAP, B7H3, Kit, CA LX, CS-1, MUC1, BCMA, bcr-abl, HER2, β-human chorionic gonadotropin, alphafetoprotein (AFP), ALK, CD19, TIM3, cyclin BI, lectin-reactive AFP, Fos-related antigen 1, ADRB3, thyroglobulin, EphA2, RAGE-1, RUI, RU2, SSX2, AKAP-4, LCK, OY-TESI, PAX5, SART3, CLL-1, fucosyl GM1, GloboH, MN-CA IX, EPCAM, EVT6-AML, TGS5, human telomerase reverse transcriptase, plysialic acid, PLAC1, RUI, RU2 (AS), intestinal carboxyl esterase, lewisY, sLe, LY6K, mut hsp70-2, M-CSF, MYCN, RhoC, TRP-2, CYPIBI, BORIS, prostase, prostate-specific antigen (PSA), PAX3, PAP, NY-ESO-1, LAGE-Ia, LMP2, NCAM, p53, p53 mutant, Ras mutant, gplOO, prostein, OR51E2, PANX3, PSMA, PSCA, Her2/neu, hTERT, HMWMAA, HAVCR1, VEGFR2, PDGFR-beta, survivin and telomerase, legumain, HPV E6, E7, sperm protein 17, SSEA-4, tyrosinase, TARP, WT1, prostate-carcinoma tumor antigen-1 (PCTA-1), ML-IAP, MAGE, MAGE-A1, MAD-CT-1, MAD-CT-2, MelanA/MART 1, XAGE1, ELF2M, ERG (TMPRSS2 ETS fusion gene), NA17, neutrophil elastase, sarcoma translocation breakpoints, NY-BR-1, ephnnB2, CD20, CD22, CD24, CD30, TIM3, CD38, CD44v6, CD97, CD171, CD179a, androgen receptor, FAP, insulin growth factor (IGF)-I, IGFII, IGF-I receptor, GD2, o-acetyl-GD2, GD3, GM3, GPRC5D, GPR20, CXORF61, folate receptor (FRa), folate receptor beta, ROR1, Flt3, TAG72, TN Ag, Tie 2, TEM1, TEM7R, CLDN6, TSHR, UPK2, and mesothelin. In a preferred embodiment, the tumor antigen is selected from the group consisting of folate receptor (FRa), mesothelin, EGFRvIII, IL-13Ra, CD123, CD19, TIM3, BCMA, GD2, CLL-1, CA-IX, MUCI, HER2, and any combination thereof.

Non-limiting examples of tumor antigens include the following: Differentiation antigens such as tyrosinase, TRP-1, TRP-2 and tumor-specific multilineage antigens such as MAGE-1, MAGE-3, BAGE, GAGE-1, GAGE-2, pi 5; overexpressed embryonic antigens such as CEA; overexpressed oncogenes and mutated tumor-suppressor genes such as p53, Ras, HER-2/neu; unique tumor antigens resulting from chromosomal translocations; such as BCR-ABL, E2A-PRL, H4-RET, IGH-IGK, MYL-RAR; and viral antigens, such as the Epstein Barr virus antigens EBVA and the human papillomavirus (HPV) antigens E6 and E7. Other large, protein-based antigens include TSP-180, MAGE-4, MAGE-5, MAGE-6, RAGE, NY-ESO, pI85erbB2, pI80erbB-3, c-met, nm-23H1, PSA, CA 19-9, CA 72-4, CAM 17.1, NuMa, K-ras, beta-Catenin, CDK4, Mum-1, p 15, p 16, 43-9F, 5T4, 791Tgp72, alpha-fetoprotein, beta-HCG, BCA225, BTAA, CA 125, CA 15-3\CA 27.29\BCAA, CA 195, CA 242, CA-50, CAM43, CD68\P1, CO-029, FGF-5, G250, Ga733\EpCAM, HTgp-175, M344, MA-50, MG7-Ag, MOV18, NB/70K, NY-CO-1, RCASI, SDCCAG1 6, TA-90\Mac-2 binding protein\cyclophilm C-associated protein, TAAL6, TAG72, TLP, TPS, GPC3, MUC16, LMP1, EBMA-1, BARF-1, CS1, CD319, HER1, B7H6, L1CAM, IL6, and MET.

### Nucleic Acids and Vectors

Also disclosed are polynucleotides and polynucleotide vectors encoding the disclosed chimeric receptors. Also disclosed are oligonucleotides for use in inserting the chimeric receptors into the genome of a T cell at a site that will disrupt Sirt2 expression or activity.

Nucleic acid sequences encoding the disclosed chimeric receptors, and regions thereof, can be obtained using recombinant methods known in the art, such as, for example by screening libraries from cells expressing the gene, by deriving the gene from a vector known to include the same, or by isolating directly from cells and tissues containing the same, using standard techniques. Alternatively, the gene of interest can be produced synthetically, rather than cloned.

### Immune Effector Cells

Also disclosed are immune effector cells that are engineered to express the disclosed chimeric receptors. These cells are preferably obtained from the subject to be treated (i.e. are autologous). However, in some embodiments, immune effector cell lines or donor effector cells (allogeneic) are used. Immune effector cells can be obtained from a number of sources, including peripheral blood mononuclear cells, bone marrow, lymph node tissue, cord blood, thymus tissue, tissue from a site of infection, ascites, pleural effusion, spleen tissue, and tumors. Immune effector cells can be obtained from blood collected from a subject using any number of techniques known to the skilled artisan, such as Ficoll™ separation. For example, cells from the circulating blood of an individual may be obtained by apheresis. In some embodiments, immune effector cells are isolated from peripheral blood lymphocytes by lysing the red blood cells and depleting the monocytes, for example, by centrifugation through a PERCOLL™ gradient or by counterflow centrifugal elutriation. A specific subpopulation of immune effector cells can be further isolated by positive or negative selection techniques. For example, immune effector cells can be isolated using a combination of antibodies directed to surface markers unique to the positively selected cells, e.g., by incubation with antibody-conjugated beads for a time period sufficient for positive selection of the desired immune effector cells. Alternatively, enrichment of immune effector cells population can be accomplished by negative selection using a combination of antibodies directed to surface markers unique to the negatively selected cells.

In some embodiments, the immune effector cells comprise any leukocyte involved in defending the body against infectious disease and foreign materials. For example, the immune effector cells can comprise lymphocytes, monocytes, macrophages, dendritic cells, mast cells, neutrophils, basophils, eosinophils, or any combinations thereof. For example, the immune effector cells can comprise T lymphocytes.

T cells or T lymphocytes can be distinguished from other lymphocytes, such as B cells and natural killer cells (NK cells), by the presence of a T-cell receptor (TCR) on the cell surface. They are called T cells because they mature in the thymus (although some also mature in the tonsils). There are several subsets of T cells, each with a distinct function.

T helper cells (T_{H} cells) assist other white blood cells in immunologic processes, including maturation of B cells into plasma cells and memory B cells, and activation of cytotoxic T cells and macrophages. These cells are also known as CD4+ T cells because they express the CD4 glycoprotein on their surface. Helper T cells become activated when they are presented with peptide antigens by MHC class II molecules, which are expressed on the surface of antigen-presenting cells (APCs). Once activated, they divide rapidly and secrete small proteins called cytokines that regulate or assist in the active immune response. These cells can differentiate into one of several subtypes, including T_{H}1, T_{H}2, T_{H}3, T_{H}17, T_{H}9, or T_{FH}, which secrete different cytokines to facilitate a different type of immune response.

Cytotoxic T cells (Tc cells, or CTLs) destroy virally infected cells and tumor cells, and are also implicated in transplant rejection. These cells are also known as CD8+ T cells since they express the CD8 glycoprotein at their surface. These cells recognize their targets by binding to antigen associated with MHC class I molecules, which are present on the surface of all nucleated cells. Through IL-10, adenosine and other molecules secreted by regulatory T cells, the CD8+ cells can be inactivated to an anergic state, which prevents autoimmune diseases.

Memory T cells are a subset of antigen-specific T cells that persist long-term after an infection has resolved. They quickly expand to large numbers of effector T cells upon re-exposure to their cognate antigen, thus providing the immune system with “memory” against past infections. Memory cells may be either CD4⁺ or CD8⁺. Memory T cells typically express the cell surface protein CD45RO.

Regulatory T cells (T_{reg} cells), formerly known as suppressor T cells, are crucial for the maintenance of immunological tolerance. Their major role is to shut down T cell-mediated immunity toward the end of an immune reaction and to suppress auto-reactive T cells that escaped the process of negative selection in the thymus. Two major classes of CD4⁺ T_{reg} cells have been described—naturally occurring T_{reg} cells and adaptive T_{reg} cells.

Natural killer T (NKT) cells (not to be confused with natural killer (NK) cells) bridge the adaptive immune system with the innate immune system. Unlike conventional T cells that recognize peptide antigens presented by major histocompatibility complex (MHC) molecules, NKT cells recognize glycolipid antigen presented by a molecule called CD1d.

In some embodiments, the T cells comprise a mixture of CD4+ cells. In other embodiments, the T cells are enriched for one or more subsets based on cell surface expression. For example, in some cases, the T comprise are cytotoxic CD8⁺ T lymphocytes. In some embodiments, the T cells comprise γδ T cells, which possess a distinct T-cell receptor (TCR) having one γ chain and one δ chain instead of a and β chains.

Natural-killer (NK) cells are CD56⁺CD3⁻ large granular lymphocytes that can kill virally infected and transformed cells, and constitute a critical cellular subset of the innate immune system (Godfrey J, et al. Leuk Lymphoma 2012 53:1666-1676). Unlike cytotoxic CD8⁺ T lymphocytes, NK cells launch cytotoxicity against tumor cells without the requirement for prior sensitization, and can also eradicate MHC-I-negative cells (Narni-Mancinelli E, et al. Int Immunol 2011 23:427-431). NK cells are safer effector cells, as they may avoid the potentially lethal complications of cytokine storms (Morgan R A, et al. Mol Ther 2010 18:843-851), tumor lysis syndrome (Porter D L, et al. N Engl J Med 2011 365:725-733), and on-target, off-tumor effects. Although NK cells have a well-known role as killers of cancer cells, and NK cell impairment has been extensively documented as crucial for progression of MM (Godfrey J, et al. Leuk Lymphoma 2012 53:1666-1676; Fauriat C, et al. Leukemia 2006 20:732-733), the means by which one might enhance NK cell-mediated anti-MM activity has been largely unexplored prior to the disclosed CARs.

### Therapeutic Methods

Immune effector cells expressing the disclosed chimeric receptors can elicit an anti-tumor immune response against cancer cells. The anti-tumor immune response elicited by the disclosed chimeric cells may be an active or a passive immune response. In addition, the immune response may be part of an adoptive immunotherapy approach in which chimeric cells induce an immune response specific to the target antigen.

Adoptive transfer of immune effector cells expressing chimeric receptors is a promising anti-cancer therapeutic. Following the collection of a patient's immune effector cells, the cells may be genetically engineered to express the disclosed chimeric receptors while ablating Sirt2 according to the disclosed methods, then infused back into the patient.

The disclosed chimeric effector cells may be administered either alone, or as a pharmaceutical composition in combination with diluents and/or with other components such as IL-2, IL-15, or other cytokines or cell populations. Briefly, pharmaceutical compositions may comprise a target cell population as described herein, in combination with one or more pharmaceutically or physiologically acceptable carriers, diluents or excipients. Such compositions may comprise buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); and preservatives. Compositions for use in the disclosed methods are in some embodiments formulated for intravenous administration. Pharmaceutical compositions may be administered in any manner appropriate treat tumors. The quantity and frequency of administration will be determined by such factors as the condition of the patient, and the severity of the patient's disease, although appropriate dosages may be determined by clinical trials.

When “an immunologically effective amount”, “an anti-tumor effective amount”, “an tumor-inhibiting effective amount”, or “therapeutic amount” is indicated, the precise amount of the compositions of the present invention to be administered can be determined by a physician with consideration of individual differences in age, weight, tumor size, extent of infection or metastasis, and condition of the patient (subject). It can generally be stated that a pharmaceutical composition comprising the T cells described herein may be administered at a dosage of 10⁴ to 10⁹ cells/kg body weight, such as 10⁵ to 10⁶ cells/kg body weight, including all integer values within those ranges. T cell compositions may also be administered multiple times at these dosages. The cells can be administered by using infusion techniques that are commonly known in immunotherapy (see, e.g., Rosenberg et al., New Eng. J. of Med. 319:1676, 1988). The optimal dosage and treatment regime for a particular patient can readily be determined by one skilled in the art of medicine by monitoring the patient for signs of disease and adjusting the treatment accordingly.

In certain embodiments, it may be desired to administer activated T cells to a subject and then subsequently re-draw blood (or have an apheresis performed), activate T cells therefrom according to the disclosed methods, and reinfuse the patient with these activated and expanded T cells. This process can be carried out multiple times every few weeks. In certain embodiments, T cells can be activated from blood draws of from 10 cc to 400 cc. In certain embodiments, T cells are activated from blood draws of 20 cc, 30 cc, 40 cc, 50 cc, 60 cc, 70 cc, 80 cc, 90 cc, or 100 cc. Using this multiple blood draw/multiple reinfusion protocol may serve to select out certain populations of T cells.

The administration of the disclosed compositions may be carried out in any convenient manner, including by injection, transfusion, or implantation. The compositions described herein may be administered to a patient subcutaneously, intradermally, intratumorally, intranodally, intramedullary, intramuscularly, by intravenous (i.v.) injection, or intraperitoneally. In some embodiments, the disclosed compositions are administered to a patient by intradermal or subcutaneous injection. In some embodiments, the disclosed compositions are administered by i.v. injection. The compositions may also be injected directly into a tumor, lymph node, or site of infection.

In certain embodiments, the disclosed chimeric cells are administered to a patient in conjunction with (e.g., before, simultaneously or following) any number of relevant treatment modalities, including but not limited to thalidomide, dexamethasone, bortezomib, and lenalidomide. In further embodiments, the chimeric cells may be used in combination with chemotherapy, radiation, immunosuppressive agents, such as cyclosporin, azathioprine, methotrexate, mycophenolate, and FK506, antibodies, or other immunoablative agents such as CAM PATH, anti-CD3 antibodies or other antibody therapies, cytoxin, fludaribine, cyclosporin, FK506, rapamycin, mycophenolic acid, steroids, FR901228, cytokines, and irradiation. In some embodiments, the CAR-modified immune effector cells are administered to a patient in conjunction with (e.g., before, simultaneously or following) bone marrow transplantation, T cell ablative therapy using either chemotherapy agents such as, fludarabine, external-beam radiation therapy (XRT), cyclophosphamide, or antibodies such as OKT3 or CAMPATH. In another embodiment, the cell compositions of the present invention are administered following B-cell ablative therapy such as agents that react with CD20, e.g., Rituxan. For example, in some embodiments, subjects may undergo standard treatment with high dose chemotherapy followed by peripheral blood stem cell transplantation. In certain embodiments, following the transplant, subjects receive an infusion of the expanded immune cells of the present invention. In an additional embodiment, expanded cells are administered before or following surgery.

The cancer of the disclosed methods can be any cell in a subject undergoing unregulated growth, invasion, or metastasis. In some aspects, the cancer can be any neoplasm or tumor for which radiotherapy is currently used. Alternatively, the cancer can be a neoplasm or tumor that is not sufficiently sensitive to radiotherapy using standard methods. Thus, the cancer can be a sarcoma, lymphoma, leukemia, carcinoma, blastoma, or germ cell tumor. A representative but non-limiting list of cancers that the disclosed compositions can be used to treat include lymphoma, B cell lymphoma, T cell lymphoma, mycosis fungoides, Hodgkin's Disease, myeloid leukemia, bladder cancer, brain cancer, nervous system cancer, head and neck cancer, squamous cell carcinoma of head and neck, kidney cancer, lung cancers such as small cell lung cancer and non-small cell lung cancer, neuroblastoma/glioblastoma, ovarian cancer, pancreatic cancer, prostate cancer, skin cancer, liver cancer, melanoma, squamous cell carcinomas of the mouth, throat, larynx, and lung, endometrial cancer, cervical cancer, cervical carcinoma, breast cancer, epithelial cancer, renal cancer, genitourinary cancer, pulmonary cancer, esophageal carcinoma, head and neck carcinoma, large bowel cancer, hematopoietic cancers; testicular cancer; colon and rectal cancers, prostatic cancer, and pancreatic cancer.

The disclosed chimeric cells can be used in combination with any compound, moiety or group which has a cytotoxic or cytostatic effect. Drug moieties include chemotherapeutic agents, which may function as microtubulin inhibitors, mitosis inhibitors, topoisomerase inhibitors, or DNA intercalators, and particularly those which are used for cancer therapy.

The disclosed chimeric cells can be used in combination with a checkpoint inhibitor. The two known inhibitory checkpoint pathways involve signaling through the cytotoxic T-lymphocyte antigen-4 (CTLA-4) and programmed-death 1 (PD-1) receptors. These proteins are members of the CD28-B7 family of cosignaling molecules that play important roles throughout all stages of T cell function. The PD-1 receptor (also known as CD279) is expressed on the surface of activated T cells. Its ligands, PD-L1 (B7-H1; CD274) and PD-L2 (B7-DC; CD273), are expressed on the surface of APCs such as dendritic cells or macrophages. PD-L1 is the predominant ligand, while PD-L2 has a much more restricted expression pattern. When the ligands bind to PD-1, an inhibitory signal is transmitted into the T cell, which reduces cytokine production and suppresses T-cell proliferation. Checkpoint inhibitors include, but are not limited to antibodies that block PD-1 (Nivolumab (BMS-936558 or MDX1106), CT-011, MK-3475), PD-L1 (MDX-1105 (BMS-936559), MPDL3280A, MSB0010718C), PD-L2 (rHlgM12B7), CTLA-4 (Ipilimumab (MDX-010), Tremelimumab (CP-675,206)), IDO, B7-H3 (MGA271), B7-H4, TIM3, LAG-3 (BMS-986016).

Human monoclonal antibodies to programmed death 1 (PD-1) and methods for treating cancer using anti-PD-1 antibodies alone or in combination with other immunotherapeutics are described in U.S. Pat. No. 8,008,449, which is incorporated by reference for these antibodies. Anti-PD-L1 antibodies and uses therefor are described in U.S. Pat. No. 8,552,154, which is incorporated by reference for these antibodies. Anticancer agent comprising anti-PD-1 antibody or anti-PD-L1 antibody are described in U.S. Pat. No. 8,617,546, which is incorporated by reference for these antibodies.

In some embodiments, the PDL1 inhibitor comprises an antibody that specifically binds PDL1, such as BMS-936559 (Bristol-Myers Squibb) or MPDL3280A (Roche). In some embodiments, the PD1 inhibitor comprises an antibody that specifically binds PD1, such as lambrolizumab (Merck), nivolumab (Bristol-Myers Squibb), or MEDI4736 (AstraZeneca). Human monoclonal antibodies to PD-1 and methods for treating cancer using anti-PD-1 antibodies alone or in combination with other immunotherapeutics are described in U.S. Pat. No. 8,008,449, which is incorporated by reference for these antibodies. Anti-PD-L1 antibodies and uses therefor are described in U.S. Pat. No. 8,552,154, which is incorporated by reference for these antibodies. Anticancer agent comprising anti-PD-1 antibody or anti-PD-L1 antibody are described in U.S. Pat. No. 8,617,546, which is incorporated by reference for these antibodies.

The disclosed chimeric cells can be used in combination with other cancer immunotherapies. There are two distinct types of immunotherapy: passive immunotherapy uses components of the immune system to direct targeted cytotoxic activity against cancer cells, without necessarily initiating an immune response in the patient, while active immunotherapy actively triggers an endogenous immune response. Passive strategies include the use of the monoclonal antibodies (mAbs) produced by B cells in response to a specific antigen. The development of hybridoma technology in the 1970s and the identification of tumor-specific antigens permitted the pharmaceutical development of mAbs that could specifically target tumor cells for destruction by the immune system. Thus far, mAbs have been the biggest success story for immunotherapy; the top three best-selling anticancer drugs in 2012 were mAbs. Among them is rituximab (Rituxan, Genentech), which binds to the CD20 protein that is highly expressed on the surface of B cell malignancies such as non-Hodgkin's lymphoma (NHL). Rituximab is approved by the FDA for the treatment of NHL and chronic lymphocytic leukemia (CLL) in combination with chemotherapy. Another important mAb is trastuzumab (Herceptin; Genentech), which revolutionized the treatment of HER2 (human epidermal growth factor receptor 2)-positive breast cancer by targeting the expression of HER2.

Generating optimal “killer” CD8 T cell responses also requires T cell receptor activation plus co-stimulation, which can be provided through ligation of tumor necrosis factor receptor family members, including OX40 (CD134) and 4-1BB (CD137). OX40 is of particular interest as treatment with an activating (agonist) anti-OX40 mAb augments T cell differentiation and cytolytic function leading to enhanced anti-tumor immunity against a variety of tumors.

In some embodiments, such an additional therapeutic agent may be selected from an antimetabolite, such as methotrexate, 6-mercaptopurine, 6-thioguanine, cytarabine, fludarabine, 5-fluorouracil, decarbazine, hydroxyurea, asparaginase, gemcitabine or cladribine.

In some embodiments, such an additional therapeutic agent may be selected from an alkylating agent, such as mechlorethamine, thioepa, chlorambucil, melphalan, carmustine (BSNU), lomustine (CCNU), cyclophosphamide, busulfan, dibromomannitol, streptozotocin, dacarbazine (DTIC), procarbazine, mitomycin C, cisplatin and other platinum derivatives, such as carboplatin.

In some embodiments, such an additional therapeutic agent is a targeted agent, such as ibrutinib or idelalisib.

In some embodiments, such an additional therapeutic agent is an epigenetic modifier such as azacitdine or vidaza.

In some embodiments, such an additional therapeutic agent may be selected from an anti-mitotic agent, such as taxanes, for instance docetaxel, and paclitaxel, and vinca alkaloids, for instance vindesine, vincristine, vinblastine, and vinorelbine.

In some embodiments, such an additional therapeutic agent may be selected from a topoisomerase inhibitor, such as topotecan or irinotecan, or a cytostatic drug, such as etoposide and teniposide.

In some embodiments, such an additional therapeutic agent may be selected from a growth factor inhibitor, such as an inhibitor of ErbBI (EGFR) (such as an EGFR antibody, e.g. zalutumumab, cetuximab, panitumumab or nimotuzumab or other EGFR inhibitors, such as gefitinib or erlotinib), another inhibitor of ErbB2 (HER2/neu) (such as a HER2 antibody, e.g. trastuzumab, trastuzumab-DM I or pertuzumab) or an inhibitor of both EGFR and HER2, such as lapatinib).

In some embodiments, such an additional therapeutic agent may be selected from a tyrosine kinase inhibitor, such as imatinib (Glivec, Gleevec STI571) or lapatinib. Therefore, in some embodiments, a disclosed antibody is used in combination with ofatumumab, zanolimumab, daratumumab, ranibizumab, nimotuzumab, panitumumab, hu806, daclizumab (Zenapax), basiliximab (Simulect), infliximab (Remicade), adalimumab (Humira), natalizumab (Tysabri), omalizumab (Xolair), efalizumab (Raptiva), and/or rituximab.

In some embodiments, a therapeutic agent for use in combination with chimeric cells for treating the disorders as described above may be an anti-cancer cytokine, chemokine, or combination thereof. Examples of suitable cytokines and growth factors include IFNy, IL-2, IL-4, IL-6, IL-7, IL-10, IL-12, IL-13, IL-15, IL-18, IL-23, IL-24, IL-27, IL-28a, IL-28b, IL-29, KGF, IFNa (e.g., INFa2b), IFN, GM-CSF, CD40L, Flt3 ligand, stem cell factor, ancestim, and TNFa. Suitable chemokines may include Glu-Leu-Arg (ELR)-negative chemokines such as IP-10, MCP-3, MIG, and SDF-Ia from the human CXC and C-C chemokine families. Suitable cytokines include cytokine derivatives, cytokine variants, cytokine fragments, and cytokine fusion proteins.

In some embodiments, a therapeutic agent for use in combination with chimeric cells for treating the disorders as described above may be a cell cycle control/apoptosis regulator (or “regulating agent”). A cell cycle control/apoptosis regulator may include molecules that target and modulate cell cycle control/apoptosis regulators such as (i) cdc-25 (such as NSC 663284), (ii) cyclin-dependent kinases that overstimulate the cell cycle (such as flavopiridol (L868275, HMR1275), 7-hydroxystaurosporine (UCN-01, KW-2401), and roscovitine (R-roscovitine, CYC202)), and (iii) telomerase modulators (such as BIBR1532, SOT-095, GRN163 and compositions described in for instance U.S. Pat. Nos. 6,440,735 and 6,713,055). Non-limiting examples of molecules that interfere with apoptotic pathways include TNF-related apoptosis-inducing ligand (TRAIL)/apoptosis-2 ligand (Apo-2L), antibodies that activate TRAIL receptors, IFNs, and anti-sense Bcl-2.

In some embodiments, a therapeutic agent for use in combination with chimeric cells for treating the disorders as described above may be a hormonal regulating agent, such as agents useful for anti-androgen and anti-estrogen therapy. Examples of such hormonal regulating agents are tamoxifen, idoxifene, fulvestrant, droloxifene, toremifene, raloxifene, diethylstilbestrol, ethinyl estradiol/estinyl, an antiandrogene (such as flutaminde/eulexin), a progestin (such as such as hydroxyprogesterone caproate, medroxy-progesterone/provera, megestrol acepate/megace), an adrenocorticosteroid (such as hydrocortisone, prednisone), luteinizing hormone-releasing hormone (and analogs thereof and other LHRH agonists such as buserelin and goserelin), an aromatase inhibitor (such as anastrazole/arimidex, aminoglutethimide/cytraden, exemestane) or a hormone inhibitor (such as octreotide/sandostatin).

In some embodiments, a therapeutic agent for use in combination with chimeric cells for treating the disorders as described above may be an anti-cancer nucleic acid or an anti-cancer inhibitory RNA molecule.

Combined administration, as described above, may be simultaneous, separate, or sequential. For simultaneous administration the agents may be administered as one composition or as separate compositions, as appropriate.

In some embodiments, the disclosed chimeric cells are administered in combination with radiotherapy. Radiotherapy may comprise radiation or associated administration of radiopharmaceuticals to a patient is provided. The source of radiation may be either external or internal to the patient being treated (radiation treatment may, for example, be in the form of external beam radiation therapy (EBRT) or brachytherapy (BT)). Radioactive elements that may be used in practicing such methods include, e.g., radium, cesium-137, iridium-192, americium-241, gold-198, cobalt-57, copper-67, technetium-99, iodide-123, iodide-131, and indium-111.

In some embodiments, the disclosed chimeric cells are administered in combination with surgery.

In some embodiments, the methods comprise administering sotorasib in an amount ranging from 240 mg to 960 mg. In some embodiments, the methods comprise administering 960 mg sotorasib to the patient once daily. In some embodiments, the methods comprise administering 240 mg to the patient once daily. In some embodiments, the methods comprise administering 480 mg to the patient twice daily. In some embodiments, the methods comprise administering 240 mg to the patient twice daily.

In some embodiments, the methods comprise administering trametinib in an amount ranging from 0.5 mg-2 mg (e.g., 0.5 mg, 0.6 mg, 0.7 mg, 0.8 mg, 0.9 mg, 1 mg, 1.1 mg, 1.2 mg, 1.3 mg, 1.5 mg, 1.6 mg, 1.7 mg, 1.8 mg, 1.9 mg, or 2 mg) In some embodiments, the methods comprise administering 1 mg trametinib to the patient. In some embodiments, the methods comprise 2 mg trametinib to the patient. In some embodiments, 0.5 mg trametinib to the patient.

In some embodiments, the methods comprise orally administering 960 mg sotorasib and trametinib in an amount ranging from 0.5 mg-2 mg (e.g., 0.5 mg, 0.6 mg, 0.7 mg, 0.8 mg, 0.9 mg, 1 mg, 1.1 mg, 1.2 mg, 1.3 mg, 1.5 mg, 1.6 mg, 1.7 mg, 1.8 mg, 1.9 mg, or 2 mg) once daily to the patient. In some embodiments, the methods comprise orally administering 960 mg sotorasib and 0.5 mg trametinib once daily to the patient. In some embodiments, the methods comprise orally administering 960 mg sotorasib and 1 mg trametinib once daily to the patient. In some embodiments, the methods comprise orally administering 960 mg sotorasib and 2 mg trametinib once daily to the patient. In some embodiments, the treatment cycle is at least 21 days. In some embodiments, the treatment cycle is 28 days or less. In some embodiments, the treatment cycle is between 21 and 28 days in length. In some embodiments, the treatment cycle is 21 days, 22 days, 23 days, 24 days, 25 days, 26 days, 27 days or 28 days in length. In some embodiments, the treatment cycle is 28 days. In some embodiments, the patient is treated for one or more treatment cycles. In various embodiments, a patient can undergo two or more treatment cycles, e.g., 2, 3, 4, 5, 6, 7, 8, 9, or 10 or more treatment cycles, or more specifically 1 to 20, 2 to 15, or 2 to 10 treatment cycles, depending upon the response of the patient to the treatment and the consideration of the attending clinician.

In some embodiments, the methods described herein further comprise administering panitumumab to the patient once every two weeks. In some embodiments, the methods further comprise administering panitumumab in an amount ranging from 3.6 mg/kg to 6 mg/kg (e.g., 3.6 mg/kg, 3.7 mg/kg, 3.8 mg/kg, 3.9 mg/kg, 4.0 mg/kg, 4.1 mg/kg, 4.2 mg/kg, 4.3 mg/kg, 4.4 mg/kg, 4.5 mg/kg, 4.6 mg/kg, 4.7 mg/kg, 4.8 mg/kg, 4.9 mg/kg, 5 mg/kg, 5.1 mg·kg, 5.2 mg/kg, 5.3 mg/kg, 5.4 mg/kg, 5.5 mg/kg, 5.6 mg/kg, 5.7 mg/kg, 5.8 mg/kg, 5.9 mg/kg, or 6 mg/kg) via IV administration once every two weeks. In some embodiments, In some embodiments, the methods further comprise administering 6 mg/kg panitumumab. In some embodiments, the methods further comprise administering 4.8 mg/kg panitumumab. In some embodiments, the methods further comprise administering 3.6 mg/kg panitumumab.

In some embodiments, the methods described herein comprise administering (a) 960 mg sotorasib orally and trametinib in an amount ranging from 0.5 mg-2 mg (e.g., 0.5 mg, 0.6 mg, 0.7 mg, 0.8 mg, 0.9 mg, 1 mg, 1.1 mg, 1.2 mg, 1.3 mg, 1.5 mg, 1.6 mg, 1.7 mg, 1.8 mg, 1.9 mg, or 2 mg) once daily to the patient; and (b) panitumumab in an amount ranging from 3.6 mg/kg to 6 mg/kg (e.g., 3.6 mg/kg, 3.7 mg/kg, 3.8 mg/kg, 3.9 mg/kg, 4.0 mg/kg, 4.1 mg/kg, 4.2 mg/kg, 4.3 mg/kg, 4.4 mg/kg, 4.5 mg/kg, 4.6 mg/kg, 4.7 mg/kg, 4.8 mg/kg, 4.9 mg/kg, 5 mg/kg, 5.1 mg·kg, 5.2 mg/kg, 5.3 mg/kg, 5.4 mg/kg, 5.5 mg/kg, 5.6 mg/kg, 5.7 mg/kg, 5.8 mg/kg, 5.9 mg/kg, or 6 mg/kg) via IV administration once every two weeks.

In various embodiments, the patient has a cancer that was determined to have one or more cells expressing the KRAS^{G12C} mutant protein prior to administration of sotorasib as disclosed herein. Determination of KRAS^{G12C} mutant protein can be assessed as described elsewhere in this disclosure. In some embodiments, the patient administered sotorasib in the methods described herein have been previously treated with a different anti-cancer therapy, e.g., at least one—such as one, or two, or three—other systemic cancer therapy. In some embodiments, the patient had previously been treated with one other systemic cancer therapy, such that the sotorasib therapy is a second line therapy. In some embodiments, the patient had previously been treated with two other systemic cancer therapies, such that the sotorasib therapy as provided herein is a third line therapy.

[In some embodiments, the prior systemic cancer therapy is a therapy with a KRAS^{G12C} inhibitor. In certain embodiments, the patient exhibits reduced sensitivity to a therapy with a KRAS^{G12C} inhibitor. In some embodiments, the patient is resistant to a therapy with a KRAS^{G12C} inhibitor. In some embodiments, KRAS^{G12C} inhibitor is sotorasib, adagrasib, GDC-6036, D-1553, JDQ443, LY3484356, BI1823911, JAB-21822, RMC-6291, or APG-1842. In certain embodiments the KRAS^{G12C} inhibitor is sotorasib. In certain embodiments, the KRAS^{G12C} inhibitor is adagrasib. In some embodiments, the therapy is monotherapy. In one embodiment, the therapy with a KRAS^{G12C} inhibitor is sotorasib monotherapy. In another embodiment, the therapy with a KRAS^{G12C} inhibitor is monotherapy with adagrasib.

As used herein “sensitivity” refers to the way a cancer reacts to a drug, e.g., sotorasib. In exemplary aspects, “sensitivity” means “responsive to treatment” and the concepts of “sensitivity” and “responsiveness” are positively associated in that a cancer or tumor that is responsive to a drug treatment is said to be sensitive to that drug. “Sensitivity” in exemplary instances is defined according to Pelikan, Edward, Glossary of Terms and Symbols used in Pharmacology (Pharmacology and Experimental Therapeutics Department Glossary at Boston University School of Medicine), as the ability of a population, an individual or a tissue, relative to the abilities of others, to respond in a qualitatively normal fashion to a particular drug dose. The smaller the dose required producing an effect, the more sensitive is the responding system. “Sensitivity” may be measured or described quantitatively in terms of the point of intersection of a dose-effect curve with the axis of abscissal values or a line parallel to it; such a point corresponds to the dose just required to produce a given degree of effect. In analogy to this, the “sensitivity” of a measuring system is defined as the lowest input (smallest dose) required producing a given degree of output (effect). In exemplary aspects, “sensitivity” is opposite to “resistance” and the concept of “resistance” is negatively associated with “sensitivity”. For example, a cancer that is resistant to a drug treatment is either not sensitive nor responsive to that drug or was initially sensitive to the drug and is no longer sensitive upon acquiring resistance; that drug is not or no longer an effective treatment for that tumor or cancer cell.

Prior systemic cancer therapies include, but are not limited to, chemotherapies and immunotherapies. Specific contemplated prior systemic cancer therapies include, but are not limited to, anti-PD1 therapy, anti-PDL1 therapy, and platinum based chemotherapy. Some examples of anti-PD1 therapy and anti-PDL1 therapies include, but are not limited to, pembrolizumab, nivolumab, cemiplimab, tisielizumab, toripalimab, aspartalizumab, dostarlimab, retifanlimab, simtilimab, pidilizumab atezolizumab, avelumab, durvalumab, and zeluvalimab (AMG 404). In some embodiments, the anti-PD1 therapy includes, but is not limited to, balstilimab, budigalimab, cadonilimab, camrelizumab, cetrelimab, cemiplimab, dostarlimab, ezabenlimab, finotonlimab, nivolumab, penpulimab, pembrolizumab, pucotenlimab, retifanlimab, rulonilimab, sasanlimab, serplulimab, sintilimab, spartalizumab, tebotelimab, tislelizumab, toripalimab, zeluvalimab (AMG 404), and zimberelimab. In some embodiments, the anti-PD1 therapy include, but is not limited to, cemiplimab, dostarlimab, pembrolizumab, or nivolumab. In some embodiments, the anti-PD1 therapy is pembrolizumab (KEYTRUDA®). In some embodiments, the anti-PD-L1 therapy includes, but is not limited to, adebrelimab, atezolizumab, avelumab, cosibelimab, durvalumab, envafolimab, erfonrilimab, garivulimab, lodapolimab, opucolimab, sugemalimab, socazolimab, and tagitanlimab. In some embodiments, the anti-PD-L1 therapy includes, but is not limited to, atezolizumab (TECENTRIQ®), durvalumab (IMFINZI®), and avelumab (BAVENCIO®). Some examples of platinum based chemotherapies include, but are not limited to, carboplatin, oxaliplatin, cisplatin, nedaplatin, satraplatin, lobaplatin, triplatin tetranitrate, picoplatin, ProLindac, and aroplatin.

In some embodiments, the patient has previously been administered a systemic cancer therapy that is a targeted therapy if the cancer was identified to have an actionable oncogenic driver mutation in the epidermal growth factor receptor gene (EGFR), anaplastic lymphoma kinase gene (ALK), and/or ROS proto-oncogene 1 (ROS1). Targeted therapies for EGFR mutations include, but are not limited to, cetuximab, panitumumab, erlotinib, gefitinib, and afatinib. Targeted therapies for ALK mutations include, but are not limited to, crizotinib, entrectinib, lorlatinib, repotrectinib, brigatinib, alkotinib, alectinib, ensartinib, and ceritinib. Targeted therapies for ROS1 mutations include, but are not limited to, crizotinib, entrecetinib, ensartinib, alkotinib, brigatinib, taletrectinib, cabozantinib, repotrectinib, lorlatinib, and ceritinib.

In various embodiments, the patient exhibits an Eastern Cooperative Oncology Group (ECOG) performance status of 0, 1 or 2 (see, e.g., Zubrod et al., 1960). Status 0 indicates fully active and able to carry on all pre-disease performance without restriction. Status 1 indicates restricted in physically strenuous activity but ambulatory and able to carry out work of a light or sedentary nature. Status 2 indicates ambulatory and capable of all selfcare but unable to carry out any work activities; up and about more than 50% of waking hours. Status 3 indicates capable of only limited selfcare, confined to bed or chair more than 50% of waking hours. Status 4 indicates completely disabled, cannot carry on any selfcare and totally confined to bed or chair. Status 5 indicates death.

A number of embodiments of the invention have been described. Nevertheless, it will be understood that various modifications may be made without departing from the spirit and scope of the invention. Accordingly, other embodiments are within the scope of the following claims.

### EXAMPLES

### Example 1: Sotorasib, a Selective G12C K-RAS Inhibitor, Modulates the Tumor Microenvironment to Promote TNFα and IFNγ Signaling and Augment Checkpoint Inhibitor and Adoptive T Cell Therapy Response

### Background

KRAS-G12C mutation represents a highly prevalent mutation in lung and colon carcinoma with unfavorable clinical outcome. Recently, new compounds were developed that selectively bind to K-RAS with G12C mutation. While recent clinical results demonstrate activity of these compounds, their underlying mechanisms of action and impact on the antitumor immune response are not well understood.

### Methods

Here, the promising G12C-Kras inhibitor (Sotorasib/AMG-510) versus MEK inhibitor (Trametinib) was tested using multiple approaches to dissect and unravel the molecular mechanisms behind the immune remodeling of the tumor microenvironment (TME) mediated by this class of drugs.

### Results

Similar to Trametinib, Sotorasib also augments the KRAS-G12C mutant cancer cells response to TNFα by increasing TNFR1 surface expression, which in turn upregulates TNFα and IFNγ down-stream target genes (including T cells chemokines) and ultimately enhances cancer cell death. In TACE-dependent mechanism, Sotorasib inhibited TNFR1 shedding off the KRAS-G12C mutant cancer cells. Interestingly, Sotorasib significantly promoted the expansion of tumor infiltrating lymphocytes (TILs) in both CT26-G12C heterotrophic, and human T cell adoptive therapy (ACT) mouse models. In addition, it generated a “hot” tumor microenvironment with strikingly augmented T cell effector phenotype. Intriguingly, Single cell RNA sequencing of CT26-G12C tumors shows an enrichment of tumor clusters with TNFα and IFNγ as top upregulated pathways in Sotorasib-treated tumors. These results suggest that Sotorasib regulation of TNFα and IFNγ plays a crucial role in generating a more immune active TME. Consistently, Sotorasib synergized with anti-PD-1 in controlling CT26-G12C tumor growth and prolonging mouse survival. Similarly, Sotorasib combined with CAR-T adoptive cell transfer and anti-PD1 treatment enhanced their anti-tumor effect.

### CONCLUSION

These findings provide insight into the molecular basis of G12C-Kras inhibitor-mediated immune modulation in the TME and set the foundation for combinatorial regimens with checkpoint inhibitor and ACT immunotherapy to achieve optimum anti-tumor activity. Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize, or be able to ascertain using no more than routine experimentation, many equivalents to the specific embodiments of the invention described herein. Such equivalents are intended to be encompassed by the following claims.

1. A method of treating cancer comprising a KRAS-G12C mutation in a subject comprising
a) administering to the subject an effective amount of a KRAS inhibitor;
b) adoptively transferring to the subject an effective amount of an immune effector cell; and
c) administering to the subject a checkpoint inhibitor.

2. The method of claim 1, wherein the KRAS inhibitor is selected from the group consisting of AMG 510 (sotorasib, LUMAKRAS™), MRTX849 (adagrasib), ARS-3248, GDC-6036, BI 1701963, tipifarnib, and BBP-454.

3. The method of claim 2, wherein the KRAS inhibitor is sotorasib.

4. The method of claim 1, wherein the immune effector cell expresses a chimeric antigen receptor (CAR) polypeptide.

5. The method of claim 1, wherein the immune effector cell is selected from the group consisting of alpha-beta T cells, gamma-delta T cells, Natural Killer (NK) cells, Natural Killer T (NKT) cells, innate lymphoid cells (ILCs), cytokine induced killer (CIK) cells, cytotoxic T lymphocytes (CTLs), lymphokine activated killer (LAK) cells, tumor infiltrating lymphocyte (TIL), and regulatory T (Treg) cells.

6. The method of claim 1, wherein the immune effector cell is autologous.

7. The method of claim 1, wherein the subject is resistant to therapy with a KRAS^{G12C} inhibitor.

8. The method of claim 1, wherein the cancer is a solid tumor.

9. The method of claim 1, wherein the cancer is small bowel cancer, appendiceal cancer, endometrial cancer, hepatobiliary cancer, small cell lung cancer, cervical cancer, germ cell tumor, ovarian cancer, gastrointestinal neuroendocrine cancer, bladder cancer, myelodysplastic/myeloproliferative neoplasms, head and neck cancer, esophagogastric cancer, soft tissue sarcoma, mesothelioma, thyroid cancer, leukemia, or melanoma.

10. The method of claim 1, wherein the cancer is non-small cell lung cancer (NSCLC) or colorectal cancer (CRC).

11. (canceled)

12. The method of claim 1, wherein the checkpoint inhibitor comprises an anti-PD-1 antibody, anti-PD-L1 antibody, anti-CTLA-4 antibody, or a combination thereof.

13. The method of claim 1, wherein the KRAS inhibitor is administered simultaneously with the immune effector cell.

14. The method of claim 1, wherein the KRAS inhibitor is administered daily for at least 1 week prior to the immune effector cell.

15. A method for enhancing adoptive cell transfer (ACT) treatment of an immune effector cell in a subject, the method comprising co-administering to the subject a KRAS inhibitor.

16-24. (canceled)
